(19) United States
(12) Patent Application Publication
(10) Pub. No.: US 2013/0281729 A1
(43) Pub. Date: Oct. 24, 2013

(54) CONTINUOUS METHOD FOR THE CARBONYLATION OF ALCOHOLS, IN PARTICULAR OF PHENYL ALCOHOLS

(71) Applicants: Raimondo Pilia, Cagnes-sur-Mer (FR); Alexandre Trani, Carros (FR); Sabrina Marie, Revest-les-Roches (FR); Fabrice De Panthou, Nice (FR)

(72) Inventors: Raimondo Pilia, Cagnes-sur-Mer (FR); Alexandre Trani, Carros (FR); Sabrina Marie, Revest-les-Roches (FR); Fabrice De Panthou, Nice (FR)

(21) Appl. No.: 13/994,045

(22) PCT Filed: Dec. 13, 2011
(86) PCT No.: PCT/FR2011/052957

§ 371 Date: Jun. 13, 2013

(30) Foreign Application Priority Data
Dec. 13, 2010 (FR) 1004839

(51) Int. Cl. C07C 51/12

(52) U.S. Cl. CPC C07C 51/12
USPC 562/406; 562/499

(57) ABSTRACT

Organic synthesis, i.e., the synthesis of carboxylic acids by direct carbonylation of alcohols in a continuous process, and more particularly, the synthesis of phenylalkylic acids, which are synthesis intermediates useful in pharmaceutical chemistry, by direct carbonylation of phenyl alkyl alcohols.

### CROSS-REFERENCE TO RELATED APPLICATIONS

The present application is a National Stage Application of PCT International Application No. PCT/FR2011/052957 (filed on Dec. 13, 2011), under 35 U.S.C. §371, which claims priority to French Patent Application No. 1004839 (filed on Dec. 13, 2010), which are each hereby incorporated by reference in their respective entireties.

### TECHNICAL FIELD

The invention relates to organic synthesis, i.e., the synthesis of carboxylic acids by direct carbonylation of alcohols in a continuous process. It relates more particularly to the synthesis of phenylalkylic acids, which are synthesis intermediates useful in pharmaceutical chemistry, by direct carbonylation of phenyl alkyl alcohols.

### BACKGROUND

It is known that carboxylic acids, and in particular phenylalkylic acids, can be obtained using phenyl alkyl alcohols by carbonylation using pressurized CO, possibly in the presence of a catalyst.

Patent application WO 02/079134 (F. Hoffmann-La-Roche AG) describes a method for manufacturing compounds with the formula:

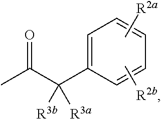

wherein R^{2a} and R^{2b} are, independently of one another, hydrogen, halogen, lower alkoxy, cyano, —COOH, lower alkoxycarbonyl, lower alkyl, and R^{3a} and R^{3b} are, independently of one another, hydrogen, lower alkyl, lower cycloalkyl, or together, —(CH₂)ₙ, with n=2, 3 or 5.

In an example, this method is used in batch mode (discontinuous) for the synthesis of 2-3,5-bis-trifluoromethylphenyl)-2-methyl-propionic acid by reaction of 2-(3,5-bis-trifluoromethyl-phenyl)-propan-2-ol with CF₃SO₃H in CH₂Cl₂ at a temperature of 20° C. and a pressure of 30 bars of CO for 170 minutes. The target molecules have a pharmaceutical interest.

This method entails two steps:

(a) the reaction of a derivative of the Grignard reagent type of a molecule of formula

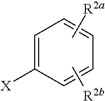

wherein X is Cl, Br or I,

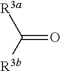

with a molecule of the type

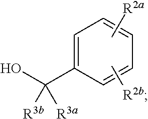

in order to form a molecule of the type:

and

b) the carbonylation of this latter molecule in the presence of a strong acid.

U.S. patent application 2007/0161814 (Sanofi-Aventis Deutschland GmbH) describes a method for manufacturing a phenylalkyl carboxylic acid or a derivative of a phenylalkyl carboxylic acid, of formula:

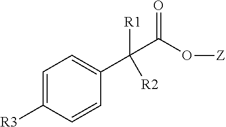

wherein R1 and R2 are independently of one another an alkyl in C₁-C₄, R3 is C(O)—(C₁-C₄) alkyl, or C(O)—(C₃-C₆) cycloalkyl, and Z is hydrogen or an alkyl in C₁-C₁₀.

The method, carried out in an autoclave, comprises the reaction of a compound of formula:

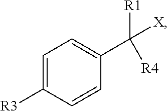

wherein X is Cl, Br or OH, R4 is R2, R1 and R3 being defined hereinabove, with CO, in the presence of an acid such as sulfuric acid or a superacid.

The article entitled “Agitated continuous mini-reactors: an industrial option for fine chemicals” by Fabrice de Panthou and Pierre Giuliano, published in Chemistry Today, vol. 26, no. 3, describes the conversion of a benzyl alcohol into the corresponding acid by direct continuous carbonylation under 40 bars of pressure of CO at approximately 50° C. in a continuous reactor: the target acid is a-methyl-4-(2-methylpropyl) benzene-ethanoic) acid, known under its pharmaceutical name ibuprofen.

There is a need for simple and effective methods that make it possible to obtain carboxylic acids with a high yield and a low rate of impurities.

### SUMMARY

The object of the invention is a continuous method of carbonylation of an alcohol referred to as “raw alcohol” into an acid referred to as the “target acid”, with the raw alcohol being: (R¹R³)C—X, wherein R¹, R³ represent radicals bonded to the carbon atom by a single covalent bond, or an aliphatic cyclic compound which incorporates the central carbon atom and which is bonded to the latter on each side by a single covalent bond; and C—X represents C(R)—OH, wherein R represents (Z¹Z²)HC— or (Z¹Z²)C—, knowing that this radical (Z¹Z²)C— can be an unsaturated cyclic compound, substituted or unsubstituted, such as a benzene cyclic compound.

The method is carried out in a piston reactor, more preferably of cylindrical shape, said reactor being provided with a mechanical axial agitation means, and in which method: continuously, more preferably at one end of said reactor, at least one liquid phase comprising said raw alcohol, possibly in an appropriate solvent, and a strong acid, are input, said at least one liquid phase subjected to mechanical axial agitation is subjected to the influence of a pressure of CO between 2 and 250 bar, and preferably between 5 and 100 bar, for a transit time t between 10 seconds and 10 minutes, preferably between 10 seconds and 6 minutes, and more preferably between 45 seconds and 4 minutes, the liquid phase is removed from said reactor, and in which method the temperature of said at least one liquid phase during the reaction is advantageously between 0° C. and 150° C., preferably between 10° C. and 100° C., and more preferably between 20° C. and 80° C., and in which method the temperature increase ΔT of the liquid between the input and the removal from the reactor is controlled in such a way that the ratio ΔT/ΔT_{ad} (where ΔT_{ad} represents the adiabatic temperature increase) is between 0.02 and 0.6 when the ratio between the characteristic heat transfer time tₜₕₑᵣₘ and the characteristic matter transfer time tₘₐₜ is between 1 and 50.

C—X can represent (R¹R³)(HZ¹Z²C)C—OH.

The target acid can correspond to the formula: R—(R¹R³)C—COOH or to the formula Z¹Z²C—(R¹R³)C—COOH.

R¹ and R³ can be, simultaneously or independently of one another, selected from the group consisting of: H; F, Cl, Br, I; an alkyl radical, straight or branched, possibly partially or totally halogenated; an aryl radical, for example phenyl, substituted or unsubstituted. R₁ and R₃ can also represent together a cycloalkyl of the type (CH₂)ₙ, substituted or unsubstituted, where n is more preferably equal to 2, 3, 4, or 5.

Z1 and Z2 can be, simultaneously or independently of one another, selected from the group consisting of: H; F, Cl, Br, I; an alkyl radical, straight or branched, possibly partially or totally halogenated; an aryl radical, for example phenyl, substituted or unsubstituted. (Z1Z2) can also represent together a cycloalkyl of the type (CH2)n, substituted or unsubstituted, where n is more preferably equal to 2, 3, 4, or 5.

The structural element (Z1Z2)C (represented by the symbol R) can be an unsaturated cyclic compound, substituted or unsubstituted, and more preferably a benzene cyclic compound or a phenyl radical mono- or poly-substituted by one or several groups selected more preferably from the group consisting of: H, F, Cl, Br, I; an alkyl radical straight or branched and possibly partially or totally substituted, more preferably by one or several atoms of halogen or one or several alkyl groups (more preferably methyl, ethyl, propyl, butyl), and more preferably by one or several radicals CF3 or C2F5.

The method according to the invention can take place only in the presence of a strong acid. This acid is advantageously selected from among: sulfuric acid, a superacid having an acidity that is stronger than concentrated sulfuric acid on the Hammet acidity scale, an acid of the Lewis type (more preferably AlCl3 or SbF5), a complex between an acid of the Lewis type and a protonic acid (more preferably the complex formed between fluorosulfonic acid HSO3F and SbF5), and more preferably selected from the group consisting of: perchloric acid, trifluoroacetic acid, fluoroantimonic acid HSb6, chlorosulfonic acid, fluorosulfonic acid, trifluoromethanesulfonic acid HSO3CF3, with the latter being particularly preferred.

Advantageously, the method according to the invention is conducted in such a way that the ratio ΔT/ΔTad is between 0.02 and 0.2 when ttherm/tmat is between 1.5 and 12, and more preferably in such a way that ΔT/ΔTad is between 0.03 and 0.15 when ttherm/tmat is between 2 and 8.

Advantageously, it is conducted in such a way that 3 s<tₘₐₜ<10 s.

### DESCRIPTION

The method according to the invention shall now be described in detail.

The raw compound (called here the “raw alcohol”) is:

(R¹R³)C—X(1) with the structural formula

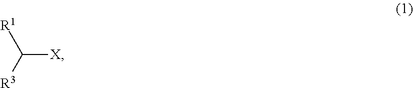

wherein R¹, R³ represent radicals bonded to the carbon atom by a single covalent bond, or an aliphatic cyclic compound which incorporates the central carbon atom and which is bonded to the latter on each side by a single covalent bond; and C—X represents C(R)—OH (2), wherein R represents

(Z¹Z²)HC— (3) with the structural formula

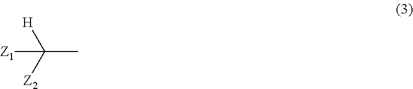

or (Z¹Z²)C— (3a), knowing that this radical (3a) can be an unsaturated cyclic compound, substituted or unsubstituted, such as a benzene cyclic compound, by way of example such a compound of type (2) is:

(R¹R³)(HZ¹Z²C)C—OH(4) with the structural formula

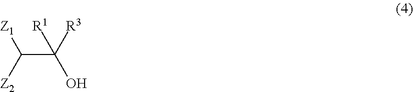

The target compound (called here the “target acid”), a product of the reaction of carbonylation according to the invention, is: R—(R1R3)C—COOH (5), or more precisely: Z1Z2C—(R1R3)C—COOH (6), knowing that in the radical Z1Z2C (3a) of this formula (6), the carbon atom C can be saturated and carry in this case an atom of hydrogen, or can be unsaturated, for example in the case where said radical (3a) is an unsaturated cyclic compound, substituted or unsubstituted, such as a benzene cyclic compound.

The inventors think, without desiring to be enclosed in this explanation, that the method according to the invention proceeds through reactions, which pass through a tertiary carbocation.

In the case where C—X represents the radical (2), the reaction could take place according to the equation (E1):

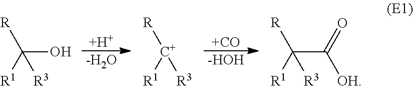

Typically, this method therefore comprises three mechanistic steps: (i) the dehydration of the alcohol in the presence of a strong acid and the formation of the carbocation; (ii) the carbonylation of the carbocation—acylium, in the presence of pressurized CO; (iii) the hydrolysis of the acylium in order to form the corresponding carboxylic acid.

The method according to the invention is a continuous method, implemented in a tubular reactor of the piston type with horizontal agitation. Consequently, these three mechanistic steps cannot be seen separately and do not correspond to sequences which can be identified in the carrying out of the method.

Information is given here on the type of compounds that can be obtained by the method according to the invention.

R1 and R3 can be, simultaneously or independently of one another:
H; F, Cl, Br, I;
an alkyl radical, straight or branched, possibly partially or totally halogenated;
an aryl radical, for example phenyl, substituted or unsubstituted.

Moreover, R1 and R3 can be part of a cycloalkyl of the type (CH2)n, substituted or unsubstituted, where n is more preferably equal to 2, 3, 4, or 5.

Z1 and Z2 can be, simultaneously or independently of one another:
H; F, Cl, Br, I;
an alkyl radical, straight or branched, possibly partially or totally halogenated;
an aryl radical, for example phenyl, substituted or unsubstituted;

knowing that:
(Z1Z2) can be a cycloalkyl of the type (CH2)n, substituted or unsubstituted, where n is more preferably equal to 2, 3, 4, or 5;
as indicated hereinabove, the structural element (Z1Z2)C (represented by the symbol R) can also be an unsaturated cyclic compound, substituted or unsubstituted, such as a benzene cyclic compound; for example R can be a mono- or poly-substituted phenyl radical, in particular with one or several atoms of a halogen or one or several alkyl groups (in particular methyl, ethyl, propyl, butyl) straight or branched and possible partially or totally substituted (for example halogenated), for example with one or several radicals CF3 or C2F5.

The method according to the invention takes place in the presence of a strong acid. Sulfuric acid can be used for example, but a superacid is preferred, i.e. an acid having an acidity H₀ that is stronger than concentrated sulfuric acid on the Hammet acidity scale, well known to those skilled in the art; an acid with an acidity H₀ of at least 12.0 is preferred. As such, perchloric acid, trifluoroacetic acid, fluoroantimonic acid HSb₆, chlorosulfonic acid, fluorosulfonic acid can be used, or more preferably trifluoromethanesulfonic acid HSO₃CF₃ (often called “triflic acid”). Acids of the Lewis type (AlCl₃, SbF₅ for example) can also be used or complexes between an acid of the Lewis type and a protonic acid (for example the complex formed between fluorosulfonic acid HSO₃F and SbF₅ which is known under the name of “magic acid”).

The method according to the invention can take place in the presence of a solvent, which contributes to the thermal dissipation of the reaction enthalpy. This solvent must be inert in relation to the strong acid and the carbonylation, and must be able to dissolve the raw alcohol sufficiently. For example a halogenated alkane (such as CH2Cl2), a chlorobenzene, an alkylbenzene (such as toluene) can be used.

The method according to the invention is carried out in a continuous reactor of the piston type reactor (also called a piston flow reactor), of length L and of volume V, wherein the chemical species (in particular the raw alcohol possibly in its solvent, the strong acid, the gaseous CO) are input at one end and are displaced all along the reactor and are progressively transformed. The reactor has more preferably a cylindrical shape. It must be provided with an axial agitation means, and more preferably with a mechanical axial agitation means. Axial agitation means here means any device that provides an agitation of the reaction mixture over the entire length, or significant portion of the latter, by a means having an axis parallel to the axis of the reactor. This axial agitation means facilitates, on the one hand, the unfolding of the reaction, by mixing the chemical species entering with the catalyst, which is in a form dispersed in a liquid phase, and facilitates on the other hand the heat transfer.

In the method according to the invention: continuously, more preferably at one end of said reactor, at least one liquid phase comprising said raw alcohol, possibly in an appropriate solvent, and a strong acid, are input, said at least one liquid phase subjected to mechanical axial agitation, in put under the influence of a pressure of CO between 2 and 250 bar, and preferably between 5 and 100 bar, for a transit time t between 10 seconds and 10 minutes, preferably between 10 seconds and 6 minutes, and more preferably between 45 seconds and 4 minutes, and the liquid phase is removed from said reactor.

The temperature of said at least one liquid phase during the reaction is advantageously between 0° C. and 150° C., preferably between 10° C. and 100° C., and more preferably between 20° C. and 80° C.

An essential characteristic of the method according to the invention is the careful control of the increase in temperature ΔT of the liquid between the input and the removal from the reactor, which must be such that the ratio ΔT/ΔTad (where ΔTad represents the adiabatic temperature increase) is between 0.02 and 0.6 when the ratio between the characteristic heat transfer time ttherm and the characteristic matter transfer time tmat is between 1 and 50.

The piston reactor has a temperature and concentration profile that can vary along its axis. Such a reactor can be modeled as a series of elementary reactors arranged in series along an axis and each having a length ΔL and a volume ΔV. In the operating conditions of this reactor, the composition of the supply and the total volume flow F are uniform and constant, and the residence time:

τ=V/F (Equation 1)

is constant for all of the molecules input into the reactor. This type of reactor is known, and those skilled in the art also know that if a highly exothermic reaction is carried out in a piston reactor, the radial heat transfer can become limiting. This is the case with carbonylation reactions.

The method according to the invention entails a chemical reaction of the type:

A(liquid)+vB(gas)→vpProduct (Equation 2),

where v is the stoichiometric coefficient of the gas and vp is the stoichiometric coefficient of the product. In accordance with the invention, the gas B is CO, and the raw alcohol to be carbonylized has the form of a pure liquid or diluted in a liquid solvent, or in the form of a solid diluted in a liquid solvent, and this liquid phase comprises a strong acid.

Generally, the performance of reactors is given by two characteristic magnitudes, which respectively describe the heat transfer and the matter transfer. These characteristic transfer times are defined hereinbelow by simplified equations (with the hydrodynamic model being the same, whether the reactor is a piston reactor or a perfectly agitated reactor, in the sense that the hydrogenation reaction is limited by the matter transfer):
the characteristic heat transfer time

$${t}_{therm} = \frac{ρ {C}_{p } {V}_{liq}}{KS} \qquad ( Equation 3 )$$

the characteristic matter transfer time

tₘₐₜ=1/(k_{L}a) (Equation 4).

In these equations, the following parameters are used: the density of the liquid p, the thermal capacity of the liquid Cp; the global transfer coefficient K, defined hereinbelow; the heat exchange surface area S (constant for a given reactor, as it is fixed through its design); and the product between the gas-liquid matter transfer coefficient on the liquid side, kL, and the specific interfacial area, a, defined hereinbelow.

The shorter the characteristic transfer time is, the better system performance is and rapidly transfers the heat and the matter (respectively).

The determination of the coefficient K well known to those skilled in the art shall now be described here in summary fashion.

The global transfer coefficient K (also called the global exchange coefficient) is defined by the equation:

φ=KSΔTₘₗ (Equation 5)

where S is the exchange surface area (in this case for a cylindrical reactor, S=πD L where D is the inside diameter and L the internal length of the portion of the tube of the reactor wherein the gas enters into contact with the liquid), ΔTml is the average logarithmic temperature difference:

ΔTml={[(T(carrier)output−T(method)input]−[(T(carrier)input−T(method)output]}/

ln{([(T(carrier)output−T(method)input]/[(T(carrier)input−T(method)output]}

and Φ is the power (in Watts, reference temperature of 25° C.) gained by the thermal flow on the method side. For a given reaction, these parameters depend on the geometry of the reactor and on the flow; they can be determined easily.

The coefficient k_{L}a, also well known to those skilled in the art, can be determined experimentally by a procedure that, in order to not needlessly complicate the description of the invention, is described hereinbelow as “Example 1”.

In an advantageous embodiment of the invention, a continuous reactor of the piston type is used that has the following characteristics:
Matter transfer: 0.1 s⁻¹<k_{LA}<0.3 s⁻¹ or 3 s<tₘₐₜ<10 s
Heat transfer: K=300 to 1000 W/m²/° C. (preferred: 700 W/m²/° C., and more preferably: about 550 W/m²/° C.) (the partial transfer coefficient of the liquid with the metal is considered here).

In a typical embodiment, the characteristic transfer time

$${t}_{therm} = \frac{ρ {C}_{p} {V}_{liq}}{KS}$$

is of the order of 25 seconds (with ρ=1050 kg/m³ Cₚ=2000 J/kg/° C.).

In this advantageous embodiment, the ratio of the characteristic times is therefore:

2<(tₜₕₑᵣₘ/tₘₐₜ)<8

In the method according to the invention, the increase in temperature of the liquid ΔT between the input and the removal of the reactor is such that:

$$\frac{Δ T}{Δ {T}_{ad}} = \frac{{t}_{therm}}{( {t}_{therm} + {τ}_{liq} )} {X}_{A} , \qquad ( Equation 6 )$$

where ΔT_{ad} is the adiabatic temperature increase:

$$Δ {T}_{ad} = \frac{( - {Δ}_{r} H ) {C}_{A 0}}{ρ {C}_{p}} , \qquad ( Equation 7 )$$

ΔᵣH is the enthalpy of the reaction, and X_{A} is the stoichiometric coefficient of the compound A.

For the case of a complete conversion of A (i.e. X_{A}=1) the equation (6) can be rewritten as:

$$\frac{Δ T}{( Δ {T}_{ad} )} = \frac{M}{( {t}_{mat} + {t}_{therm} + 1 )} , \qquad ( Equation 8 )$$

where M designates the stoichiometric ratio:

$$M = \frac{P}{{vHeC}_{A 0}} , \qquad ( Equation 9 )$$

wherein P signifies the working pressure, He signifies Henry's coefficient, C_{AO} signifies the concentration of the liquid at the input of the reactor.

The choice of the operating conditions of the method according to the invention entails three magnitudes:
the adiabatic temperature increase in an undiluted medium

$${( Δ {T}_{ad} )}_{pure} = \frac{( - {Δ}_{r} H ) {( {C}_{A 0} )}_{pure}}{ρ {C}_{p}} \qquad ( Equation 10 )$$

the stoichiometric ratio calculated on the concentration of the pure reagents

$${M}_{pure} = \frac{P}{{vHe ( {C}_{A 0} )}_{pure}} \qquad ( Equation 11 )$$

the dilution factor F defined by

$${( {C}_{A 0} )}_{working} = \frac{{( {C}_{A 0} )}_{pure}}{F} . \qquad ( Equation 12 )$$

The inventors discovered that a particular operating regime of a piston reactor makes it possible to resolve the problem in question. This regime is explained here in the case of a reaction with a stoichiometric coefficient vₚ=1, as is the case for example for the hydrogenation of ortho-cresol (used in example 1 in order to determine the k_{L}a parameter of the reactor) or the carbonylation of an alcohol. This regime relates both to a flow regime and to an agitation regime.

Indeed, according to the invention, the reaction is conducted in such a way that the increase in temperature ΔT of the liquid between the input and the removal from the reactor is such that the ratio ΔT/ΔTad (where ΔTad represents the adiabatic temperature increase) is between 0.02 and 0.6 when the ratio between the characteristic heat transfer time ttherm and the characteristic matter transfer time tmat is between 1.5 and 50. This method can be used without solvent, i.e. the mixture between said raw alcohol and the strong acid constitutes the liquid which is input into the reactor. But it is preferable to use an appropriate solvent. Most often, the liquid that is input into the reactor comprises two liquid phases: the organic phase which comprises the raw alcohol and its solvent, and the strong acid phase. The reaction proceeds in this case in a triphasic medium, with the CO gas representing the third phase. The control of the transfer of mass is therefore critical; to this end, the reactor must have an axial agitation means, which shall be described hereinbelow.

In a preferred embodiment, the ratio ΔT/ΔTad is between 0.02 and 0.2 when ttherm/tmat is between 1.5 and 12. In a more preferred embodiment, ΔT/ΔTad is between 0.03 and 0.15 when ttherm/tmat is between 2 and 8.

In order to maintain the internal temperature of the reactor constant, the heating capacity of the reaction chamber of the reactor can be adjusted, knowing that a portion of the thermal energy required to maintain it at the selected temperature comes from the enthalpy of the reaction of carbonylation which takes place in the reaction chamber.

In certain cases, it may be required to cool the reactor, in particular when the reaction temperature is between 0° C. and 30° C.

Advantageously, the method according to the invention is implemented in a tubular piston reactor of cylindrical shape with an inside diameter between 20 mm and 100 mm. Above 100 mm, the productivity of the reactor decreases because, in order to the exchange surface area to remain substantial, the flow has to be decreased. Below 20 mm, the surface/volume ratio is very high, but the flow is insufficient for industrial production. Preferably, the inside diameter of the piston reactor is between 30 mm and 75 mm, and more preferably between 40 mm and 60 mm. The length of the reaction chamber of the reactor is between 10 cm and 100 cm. Below 10 cm, the residence time is too short. Above 100 cm, the machining of the tubular reactor becomes difficult, and the agitation of the reaction mixture is difficult to accomplish. A preferred length is between 20 cm and 80 cm.

The reactor must be provided with an axial agitation means, which is more preferably a mechanical axial agitation means. Axial agitation means is understood here as any device that provides an agitation of the reaction mixture over the entire length, or significant portion of the latter, by a means that has an axis parallel to the axis of the reactor. Various means can be used for this purpose, such as a series of mixers, endless screws, a propeller, but this mechanical axial agitation means must not disturb the “piston” nature of the reactor, such as defined by the equation (1). Said axial agitation means facilitates, on the one hand, the unfolding of the reaction by mixing the chemical species that are input into the reactor, and facilitates on the other hand the heat transfer.

The continuous method can be described as comprising several steps. In a first step, continuously, more preferably at one end of said reactor, at least one liquid phase is input, comprising said raw alcohol and the strong acid. Then said at least one liquid phase is subjected to a temperature between 0° C. and 150° C. and subjected to mechanical axial agitation, and to the influence of a pressure of carbon monoxide between 1 and 200 bar (preferred: between 2 and 50 bar) for a transit time t between 1 second and 10 minutes (preferred: 10 seconds and 6 minutes, and more preferably 40 seconds to 3 minutes). When the liquid phase has arrived at the other end of the reactor, it is removed at the other end of the reactor.

The method according to the invention makes it possible to produce industrial quantities of carboxylic acids, for example of a magnitude of 2 to 20 kg/h in the case of phenylalkylic acids. This makes it possible to access an annual production of a magnitude of 20 to 100 tons with a single reactor. For a continuous reactor, this represents productivity that is entirely interesting from an industrial standpoint, even in the case of simple molecules. The cost of investment for a reactor able to implement the method according to the invention is lower than that for a reactor of the batch type, and the needs in terms of labor are reduced. The “scaling up” of the method is substantially simplified since the method according to the invention can be implemented in an industrial continuous reactor of small size, which does not differ greatly from a laboratory experimental reactor. In order to increase productivity, the diameter of the reactor can be increased, but this possibility is limited by the transfer of heat, as explained hereinabove. Advantageously, a plurality of reactors is used, in light of their simplicity, of the continuous nature of the method and of the fact that this continuous method does not require the intervention of a lot of labor.

Certain embodiments of the invention shall now be described here more precisely. In accordance with a first embodiment of the invention, the raw alcohol (1) is a molecule wherein C—X represents C(R)—OH (2) wherein R represents a mono- or poly-substituted phenyl radical, in particular via a halogen or an alkyl group, for example 2,3,4-trifluorophenyl-, 2,3,4-trichlorophenyl-, 2,3 (or 3, 4 or 4, 5 or 2, 5 or 3, 5 or 2,4))bischlorophenyl-, 2 (or 3 or 4 or 5)-chlorophenyl-, 2 (or 3 or 4 or 5)-fluorophenyl-, 2 (or 3 or 4 or 5)-methylphenyl, 2,3 (or 3,4 or 4, 5 or 2, 5 or 3, 5 or 2,4)bimethylphenyl-, and the method takes place in the following way:

(a) The raw alcohol (1) in solution in CH2Cl2 at a concentration of approximately 40% by weight, a strong acid (more preferably liquid triflic acid (trifluoromethane sulfonic acid)) and gaseous carbon monoxide under a pressure of approximately 5 to 50 bars (for example 40 to 50 bars) are introduced simultaneously into the reactor preheated to the target temperature;

(b) The axial agitation in the reactor is of a magnitude of 1400 rpm, the residence time is approximately 3 minutes, the temperature (“target temperature”) of the reaction is maintained at 40° C.-50° C. (for example approximately 45° C.), the CO pressure is constant and approximately 5 to 50 bars (for example 40 to 50 bars);

(c) At removal, the solution obtained is cooled in-line at 20-25° C., then released to atmospheric pressure, collected in a first tank for 7 minutes, then in a second tank containing water at approximately 5° C.;

(d) Finally, the solution is subjected to steps of separating and recovering known as such. By way of example: the aqueous phase containing the triflic acid is separated from the organic phase which contains the target acid, the triflic acid is recovered by distillation and recycled in the reaction, the organic phase is treated with soda in order to obtain a salt of the target acid in water then this solution is acidified in order to regenerate the acid which is extracted; it can be crystallized in an appropriate solvent.

In accordance with a second embodiment, which can be an alternative of the first, formic acid is used in addition to pressurized CO, which makes it possible in certain cases to decrease the pressure of CO and to better solubilize it. This mechanism probably entails a dehydration reaction

H—CO₂H+H⁺ [symbol] H—CO⁺+H₂O

H—CO⁺ [symbol] CO_{liq}+H⁺

resulting in CO created in situ (symbolized by CO liq) which is immediately available for the carbonylation; the soluble CO is recreated due to the equilibrium

CO_{liq} [symbol] CO_{gas}.

However, it is observed in certain cases that the use of formic acid leads to a less pure product.

### EXAMPLES

### Example 1

Indicated here is an experimental method that can be used in order to determine the parameter kLa of a piston reactor by using a reaction that is simple and perfectly known and which can be controlled well, i.e. catalytic hydrogenation of the nitrobenzene as aniline (Ph-NO2+3H2→Ph-NH2+2H20, where Ph designates a phenyl group), which however is outside of this invention.

This reaction is carried out in liquid phase without a solvent, the gas phase being constituted of pure hydrogen at an initial pressure of 2 bars. The catalyst is constituted of powdered carbon (equivalent particle diameter of the order of 50 μm) loaded to 5% by weight in palladium. The mass concentration of the catalyst is 2.5 g/l and the hydrogenation is carried out at ambient temperature. A quartz pressure sensor makes it possible to measure the pressure of hydrogen as a function of time. The reactor has a double jacket; a circulation of thermostatically controlled water inside the double jacket makes it possible to maintain the temperature of the reactor constant. At the beginning, the non-agitated reactor is maintained under the pressure of nitrogen; it is then purged with hydrogen. At a pressure of hydrogen of 2 bars, the agitation is initiated and results in the drop in the pressure of hydrogen. The reaction is allowed to continue until the pressure reaches the value of 0.5 atm. Then, the agitation is stopped and the device is re-pressurized with hydrogen, then there is a waiting period of about ten minutes and the measure is repeated with a different agitation speed. For each test carried out, it is observed that the pressure of hydrogen decreases following an exponential law. As such, by plotting ln PH2/P0=f(t), a straight line is obtained of which the slope makes it possible to access the product α kapp. If the change in this product α kapp is plotted with the agitation speed, an asymptotic behavior is observed. For low agitation speeds, the apparent conductance increases with the agitation speed; this indicates a limitation in the apparent kinetics for the gas-liquid transfer. For strong agitation speeds, a plateau is reached; this indicates that the transfer is limited, either by the chemical kinetics or by the liquid-solid matter transfer kinetics. The use of the curve α kapp=f(agitation speed) therefore makes it possible to estimate the value of the transfer conductance kLa. In practice, five to ten experimental points are sufficient to determine this value.

The theoretical bases of this determination of kLa are indicated here.

By ignoring the accumulation of the hydrogen in liquid phase, the expression of the flow of disappearance of the hydrogen in a closed reactor can be established.

$$\frac{d {n}_{{M}_{2}}}{d t} = \frac{{V}_{G}}{RT} \frac{d {P}_{{H}_{2}}}{d t} = {ϕ}_{{H}_{2}} {V}_{R} \qquad ( 1 )$$

where ΦH2 is the specific flow of the disappearance of hydrogen. This flow can be expressed by causing the reaction speed or the transfer flow to appear:

$${ϕ}_{{H}_{2}} = {r}_{V} α = {( {K}_{{H}_{2}} a )}_{global} ( \frac{{P}_{{H}_{2}}}{He} - {C}_{{H}_{2}}^{surface} ) \qquad ( 2 )$$

knowing that rv, is the volumetric reaction speed of the hydrogenation, α the solid retention in the reactor and (KH2a) global the global transfer conductance of the hydrogen of the gas phase towards the surface of the catalyst.

With the volume of the catalyst and the concentration in nitrobenzene being considered as invariable during a test, the speed of the reaction can be expressed as resulting from a first-order kinetic modeling in relation to the concentration in hydrogen, or:

rᵥ=ηkᵥC_{NS}C_{H}_{₂}^{surface}=kᵥ′C_{H}_{₂}^{surface}

When the gas phase is of pure hydrogen, the global transfer conductance can be expressed as a function of the partial gas-liquid and liquid-solid transfer conductances by:

$$\frac{1}{{[ {K}_{{H}_{2}} a ]}_{global}} = \frac{1}{{k}_{L} {a}_{LG}} + \frac{1}{{k}_{S} {a}_{S} α} \qquad ( 4 )$$

where as is the specific surface area of the solid and aLG the specific gas-liquid surface area. By combining the expressions of the chemical kinetics and of the physical kinetics, the specific flow of the disappearance of the hydrogen in the reactor can be expressed by:

$${ϕ}_{{H}_{2}} = α {k}_{app} \frac{{P}_{{H}_{2}}}{He} \qquad ( 5 )$$

where α kapp is an apparent conductance which incorporates the limitations due to the chemical kinetics, but also the limitations due to the physical kinetics.

$$\frac{1}{α {k}_{app}} = \frac{1}{{[ {K}_{{H}_{2}} a ]}_{global}} + \frac{1}{α {k}_{v}^{′}} = \frac{1}{{k}_{L} {a}_{LG}} + \frac{1}{α} [ \frac{1}{{k}_{S} {a}_{S}} + \frac{1}{{k}_{v}^{′}} ] \qquad ( 6 )$$

By injecting (5) into (1) the following is obtained:

$$\frac{d {P}_{{H}_{2}}}{d t} = - α {k}_{app} \frac{{V}_{R}}{{V}_{G}} \frac{RT}{He} dt \qquad ( 7 )$$

of which the integration results in:

$$ln \frac{{P}_{{H}_{2}}}{{P}_{0}} = - α {k}_{app} \frac{{V}_{R}}{{V}_{G}} \frac{RT}{He} ( t - {t}_{0} ) \qquad ( 8 )$$

The interpretation of the change of the pressure of hydrogen in a closed system as such makes it possible to determine the apparent conductance of the system. The latter makes it possible to return to the value of the gas-liquid transfer conductance.

### Example 2

An example embodiment of the method in accordance with the invention shall be described here in detail; this example does not limit the invention.

The reaction used is the carbonylation of the 2-(3-(trifluoromethyl)phenyl) propan-2-ol) in order to obtain the 2-(3(trifluoromethyl)phenyl)-2-methyl propionic acid.

The raw alcohol can be synthesized via treatment of the corresponding Grignard reagent with a ketone; this reaction must comply with precise conditions in order to prevent a risk of explosion.

The acid that was used to implement the method in accordance with the invention was triflic acid, at a rate of 2.5 parts in relation to pure alcohol.

The tubular reactor used is of a Hastelloy C22 alloy. It has two inputs for liquids and one input for gases.

The following operating procedure was implemented:
A solution of the raw alcohol is prepared (at a rate of 40% by weight) in CH2Cl2; this solution is preheated to the temperature of the reactor (45° C.).
This solution is injected into the reactor with a constant flow of 65 g/min (or 26 g of pure alcohol), at the same time as the triflic acid at a rate of 65 g/min (or 2.5 parts). The pressure of CO is adjusted by a regulator to 45±2 bar at removal from the bottle and maintained constant during the reaction; the consumption was of a magnitude of 7 to 9 g/min. The temperature of the reactor is maintained constant at 45±2° C., the residence time of the reaction mixture in the reactor is of a magnitude of 3 minutes. The horizontal agitation during the reaction is set to 1400 rpm. All of the flows are controlled using mass flow meters.
When removed from the reactor, the solution is cooled in-line to about 20-25° C., then released to atmospheric pressure and collected in a first tank for 7 minutes (approximately 2 times the residence time in the reactor). Finally, it is collected in a tank containing water at approximately 5° C. for hydrolysis.
the aqueous phase containing the triflic acid is separated for revalorization (distillation), the organic phase (solvent CH2Cl2 containing the target carboxylic acid) is treated with soda in order to form the sodium salt in the water. Finally, it is acidified in order to obtain the target acid which is extracted and crystallized in toluene.

This acid is of great purity. It can be used as is as a starting or intermediate point for the synthesis of other more complex molecules, in particular molecules with a pharmaceutical interest.

### Example 3

By a method similar to that described in example 2, the following acids were synthesized from their corresponding alcohol:

(a) α,α,3,5-tetramethyl-benzene-acetic acid (C12H16O2, CAS number: 93748-16-4).

(b) α,α,dimethyl-3-(trifluoromethyl)-benzene-acetic acid (C11H11F3O2, CAS number: 254895-42-6).

(c) α,α,diethyl-benzene-acetic acid (C12H16O2, CAS number: 5465-28-1).

(d) 1-adamantanecarboxylic acid (C11H16O2, CAS number: 828-51-3).

The term “corresponding alcohol” here identifies the alcohol in accordance with the formula (1) which results in the target acid in accordance with the formula (5).

In an alternative, in these two molecules (a) and (b) one or several of said methyl groups (CH3) can be replaced with an ethyl or n-propyl group, and in the molecule (c) one or several of said ethyl group with a methyl or n-propyl group.

1-10. (canceled)

11. A method of carbonylation of a raw alcohol into a target acid, the raw alcohol being:
(R¹R³)C—X (1) with the structural formula 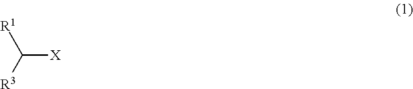
wherein:
—R¹, R³ represent radicals bonded to the carbon atom by a single covalent bond, or an aliphatic cyclic compound which incorporates a central carbon atom and which is bonded to the central carbon atom on each side by a single covalent bond; and
—C—X represents C(R)—OH (2),
wherein R represents (Z¹Z²)C— (3a) in which radical (3a) is an unsaturated cyclic compound, substituted or unsubstituted, the continuous method comprising:
continuously inputting at one end of a reactor, at least one liquid phase comprising said raw alcohol in a solvent and a strong acid;
agitating said at least one liquid phase under a pressure of CO between 5 and 100 bar, for a transit time between 45 seconds and 4 minutes;
removing the liquid phase from the reactor,
wherein a temperature of said at least one liquid phase during the reaction is between 20° C. and 80° C.,
wherein a temperature increase ΔT of the at least one liquid phase between being input and removed from the reactor is controlled in such a way that the ratio ΔT/ΔT_{ad}, where ΔT_{ad} is an adiabatic temperature increase, is between 0.02 and 0.6 when the ratio between the characteristic heat transfer time tₜₕₑᵣₘ and a characteristic matter transfer time tₘₐₜ is between 1 and 50.

12. The method of claim 11, wherein C—X represents:
(R¹R³)(HZ¹Z²C)C—OH (4) with a structural formula

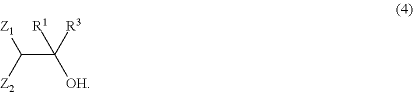

13. The method of claim 11, wherein the target acid corresponds to a formula: R—(R¹R³)C—COOH (5).

14. The method of claim 11, wherein the target acid corresponds to a formula: Z¹Z²C—(R¹R³)C—COOH (6).

15. The method of claim 11, wherein R¹ and R³ are selected from the group consisting of: H; F, Cl, Br, I, an alkyl radical, and an aryl radical.

16. The method of claim 11, wherein R¹ and R³ together represent a cycloalkyl of the type (CH₂)ₙ, substituted or unsubstituted, where n is equal to 2, 3, 4, or 5.

17. The method of claim 11, wherein Z¹ and Z² are selected from the group consisting of: H; F, Cl, Br, I, an alkyl radical, and an aryl radical.

18. The method of claim 17, wherein the structural element (Z¹Z²)C (represented by the symbol R) is a benzene cyclic compound or a mono- or poly-substituted phenyl radical with one or several groups selected from the group consisting of H; F, Cl, Br, I, an alkyl radical, one or several atoms of halogen, methyl groups, ethyl groups, propyl groups, butyl group, and one or more radicals of CF₃ or C₂F₅.

19. The method of claim 11, wherein the structural element (Z¹Z²) represent an cycloalkyl of the type (CH₂)ₙ, substituted or unsubstituted, where n is equal to 2, 3, 4, or 5.

20. The method of claim 11, wherein said strong acid comprises is selected from the group consisting of: perchloric acid, trifluoroacetic acid, fluoroantimonic acid HSb6, chlorosulfonic acid, fluorosulfonic acid, trifluoromethanesulfonic acid HSO₃CF₃.

21. The method of claim 11, wherein said strong acid comprises trifluoromethanesulfonic acid HSO₃CF₃.

22. The method of claim 11, wherein ΔT/ΔT_{ad} is between 0.02 and 0.2 when tₜₕₑᵣₘ/tₘₐₜ is between 1.5 and 12.

23. The method of claim 11, wherein ΔT/ΔT_{ad} is between 0.03 and 0.15 when tₜₕₑᵣₘ/tₘₐₜ is between 2 and 8.

24. The method of claim 11, wherein 3 s<tₘₐₜ<10 s.

25. The method of claim 11, wherein said target acid is selected from the group formed by:
a) α,α,3,5-tetramethyl-benzene-acetic acid (C₁₂H₁₆O₂, CAS number: 93748-16-4);
b) α,α,dimethyl-3-(trifluoromethyl)-benzene-acetic acid (C₁₁H₁₁F₃O₂, CAS number: 254895-42-6);
c) α,α,diethyl-benzene-acetic acid (C₁₂H₁₆O₂, CAS number: 5465-28-1);
d) 1-adamantanecarboxylic acid (C₁₁H₁₆O₂, CAS number: 828-51-3),
each of said acids being obtained from its corresponding alcohol.

26. The continuous method of claim 11, wherein the radical (3a) comprises a benzene cyclic compound.

27. The continuous method of claim 11, wherein the reactor comprises a piston reactor provided with an axial agitation mechanism.

28. A method of carbonylation of an alcohol into an acid, the alcohol being:
(R¹R³)C—X (1) with the structural formula 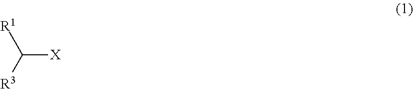
wherein:
—R¹, R³ represent radicals bonded to the carbon atom by a single covalent bond, or an aliphatic cyclic compound which incorporates a central carbon atom and which is bonded to the central carbon atom on each side by a single covalent bond; and
—C—X represents C(R)—OH (2),
wherein R represents
(Z¹Z²)HC— (3) with a structural formula 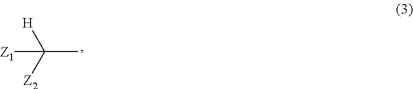
the continuous method comprising:
continuously inputting at one end of a reactor, at least one liquid phase comprising said raw alcohol in a solvent and a strong acid;
agitating said at least one liquid phase under a pressure of CO between 5 and 100 bar, for a transit time between 45 seconds and 4 minutes;
removing the liquid phase from the reactor,
wherein a temperature of said at least one liquid phase during the reaction is between 20° C. and 80° C.,
wherein a temperature increase ΔT of the at least one liquid phase between being input and removed from the reactor is controlled in such a way that the ratio ΔT/ΔT_{ad}, where ΔT_{ad} is an adiabatic temperature increase, is between 0.02 and 0.6 when the ratio between the characteristic heat transfer time tₜₕₑᵣₘ and a characteristic matter transfer time tₘₐₜ is between 1 and 50.

29. The method of claim 28, wherein said acid comprises trifluoromethanesulfonic acid HSO₃CF₃.

30. The continuous method of claim 28, wherein the reactor comprises a piston reactor provided with an axial agitation mechanism.
